(19) United States
(12) Patent Application Publication
(10) Pub. No.: US 2016/0051774 A1
(43) Pub. Date: Feb. 25, 2016

(54) OUTER CYLINDER WITH NEEDLE AND METHOD FOR PRODUCING SAME

(71) Applicant: TERUMO KABUSHIKI KAISHA, Tokyo (JP)

(72) Inventors: Tsutomu SUGIKI, Fuji-shi (JP); Kenta GOTO, Kai-shi (JP)

(73) Assignee: TERUMO KABUSHIKI KAISHA, Tokyo (JP)

(21) Appl. No.: 14/930,035

(22) Filed: Nov. 2, 2015

Related U.S. Application Data
(63) Continuation of application No. PCT/JP2014/058202, filed on Mar. 25, 2014.

(30) Foreign Application Priority Data
May 2, 2013 (JP) 2013-96931

(51) Int. Cl. A61M 5/34; B23K 26/20; B29C 65/36; B29C 65/14; B29C 65/34; B23K 26/324; B23K 13/01

(52) U.S. Cl. CPC A61M 5/343; A61M 5/349; B23K 26/324; B23K 26/20; B23K 13/01; B29C 65/1435; B29C 65/3456; B29C 65/3668; B29C 65/3676; A61M 2207/00; B23K 2203/18

(57) ABSTRACT

A method of producing a needle-mounted outer cylinder involves a melt-bond process of melting and bonding a proximal portion of a needle tube to an outer cylinder in a state in which the needle tube is in a needle insertion hole of the outer cylinder and in a state in which the outer cylinder is erect. The melt-bond process is performed by imparting energy to a portion of the needle tube distal from a proximal end of the needle insertion hole and from a proximal end of the needle tube causing the needle tube to generate heat so that resin forming an inner surface of the needle insertion hole melts and thereafter by causing the melted resin to flow downward inside the needle insertion hole to thereby fill a gap between an outer surface of the proximal portion of the needle tube and the needle insertion hole.

This application is a continuation of International Application No. PCT/JP2014/178242 filed on Mar. 25, 2014 and claims priority to Japanese Application No. 2013-96931 filed on May 2, 2013, the entire content of both of which is incorporated herein by reference.

### TECHNICAL FIELD

The present invention relates to a needle-mounted outer cylinder in which a needle tube of a syringe is directly joined with a distal end of an outer cylinder of the syringe in advance and a method of producing the needle-mounted outer cylinder.

### BACKGROUND ART

When a medicine is injected subcutaneously or intradermally for disease prevention, disease diagnosis or disease treatment, administration of various vaccines is exemplified as the disease prevention. Administration of tuberculin is exemplified as the disease diagnosis. Administration of protein preparation and antibody drug formulation such as insulin, growth hormone, erythropoietin, granulocyte colony-stimulating factor is exemplified as the disease treatment. Because the dose of these medicines is mostly small, syringes having a small capacity (a capacity not more than several milliliters and more specifically, 1 mL to 5 mL) are normally used.

In a syringe having a small capacity, there is used a needle-mounted outer cylinder in which a needle tube of a syringe is directly joined with a distal end of an outer cylinder of the syringe in advance. As methods of producing such needle-mounted outer cylinders, a method of joining the needle tube with the distal end of the outer cylinder with an adhesive agent or the like, a method of joining the needle tube with the distal portion of the outer cylinder by insert molding, and in addition, a method of heat-welding the needle tube to the outer cylinder (see patent Japanese Patent Application Laid-Open Publication Number 2004-154210) are known. In Japanese Patent Application Laid-Open Publication Number 2012-254102, there is disclosed the needle-mounted outer cylinder having the barrel 1 tubularly formed and the needle 2 fixed to the barrel 1. The barrel 1 has the tubular needle fixing part 12 to which the needle 2 inserted thereinto is bonded at the distal part thereof. The needle fixing part 12 has the welding part 12a which is thermoplastic and heated to weld the needle 2 thereto. The welding part 12a is disposed at the position axially apart from the distal end of the needle fixing part 12.

### SUMMARY

In the case where the insert molding is used as disclosed in Japanese Patent Application Laid-Open Publication Number 2004-154210, it is necessary to perform the work of disposing the needle tube having the blade surface formed thereon inside a die. There is a possibility that the blade surface may be damaged during this work. In the case where the joining member is used as disclosed in the patent document 2, it is necessary to perform the work of inserting the joining member into the distal portion of the outer cylinder and insert the needle tube into the joining member. In addition, in joining the needle tube with the joining member, it is necessary to perform the work of welding the needle tube with the joining member and heat-sealing the joining member with the outer cylinder. Therefore there is a possibility that the needle tube joining work may be complicated.

In the needle-mounted barrel of Japanese Patent Application Laid-Open Publication Number 2012-254102, the joining member is not used. The needle fixing part has the supporting portion which supports the needle radially with the needle fixing part in contact with the outer peripheral portion of the needle. The welding part is disposed at the position axially apart from the proximal end of the needle fixing part. The supporting portion is disposed at both sides of the welding part in the axial direction of the barrel. In the needle-mounted barrel of Japanese Patent Application Laid-Open Publication Number 2012-254102, it is necessary to perform the operation of inserting the needle into the small-diameter supporting portion which contacts the outer periphery of the needle. Insertion resistance is applied to the needle in the insertion operation. Thus the workability in the production process is low. In addition, in dependence on the dimension of inner diameters of portions other than the supporting portion, there is a possibility that the amount of the melted resin forming the supporting portion is insufficient for fixing the needle to the welding part.

Upon present inventors' earnest investigations, it is effective as a method of producing the needle-mounted outer cylinder of this type to apply energy to a metal needle tube from outside to allow the needle tube to generate heat and fusion-fix the needle tube to the outer cylinder by utilizing the generated heat. But the present inventors have found that in the case where the needle tube is fusion-fixed to the outer cylinder by using this method, foaming occurs at a melted portion and thus there is a possibility that foaming-caused defective fixing occurs.

The method and needle-mounted outer cylinder disclosed here has been developed in view of the above-described problems. The needle-mounted outer cylinder disclosed here allows a needle tube insertion work to be performed easily by setting into a needle insertion hole formed at a distal part of the outer cylinder larger than an outer diameter of the needle tube and which hardly generates foaming at a melt-bond portion and foaming-caused defective fixing of the needle tube to the outer cylinder, and a method of producing the needle-mounted outer cylinder.

A needle-mounted outer cylinder comprises a metal needle tube and an outer cylinder, made of thermoplastic resin, which has a distal part having a needle insertion hole; and a proximal portion of said needle tube is bonded to said outer cylinder with a melt-bond portion formed inside said needle insertion hole by melting an inner surface of said needle insertion hole. Said needle insertion hole has an inner diameter larger than an outer diameter of said proximal portion of said needle tube; said melt-bond portion is positioned at a side distal by a predetermined length from a proximal end of said needle insertion hole and is formed by partly melting said inner surface of said needle insertion hole, by flowing down toward a proximal side of said needle insertion hole and by filling a gap between an outer surface of said proximal portion of said needle tube and said inner surface of said needle insertion hole with a portion of said inner surface of said needle insertion hole; and said distal part of said outer cylinder does not substantially have bubbles to be formed when said melt-bond portion is formed.

A method of producing a needle-mounted outer cylinder comprises a metal needle tube and an outer cylinder, made of thermoplastic resin, which has a distal part having a needle insertion hole into which a proximal portion of said needle tube is inserted; and said proximal portion of said needle tube is bonded to said outer cylinder with a melt-bond portion formed inside said needle insertion hole by melting an inner surface of said needle insertion hole. Said method of producing said needle-mounted outer cylinder involves execution of a melt-bond process of melting and bonding said proximal portion of said needle tube to said outer cylinder in a state in which said needle tube is inserted into said needle insertion hole of said outer cylinder and in which said outer cylinder is erect; and said melt-bond process being performed by imparting energy to said needle tube at a portion thereof positioned at a side distal from a proximal end of said needle insertion hole and from said proximal end of said needle tube to allow said needle tube to generate heat so that resin positioned at a heat generation portion and forming said inner surface of said needle insertion hole is melted, and thereafter by flowing said melted resin downward inside said needle insertion hole to thereby fill a gap between an outer surface of said proximal portion of said needle tube and said needle insertion hole.

### BRIEF DESCRIPTION OF THE DRAWINGS

FIG. 1 is a front view of a needle-mounted outer cylinder of the present invention.

FIG. 2 is a plan view of the needle-mounted outer cylinder of the present invention.

FIG. 3 is a sectional view of the needle-mounted outer cylinder shown in FIG. 2 taken along a section line 3-3 in FIG. 2.

FIG. 4 is a main part-depicted vertical sectional view of the needle-mounted outer cylinder of the present invention.

FIG. 5 is a vertical sectional view of the outer cylinder.

FIG. 6 is an exploded perspective view of the needle-mounted outer cylinder of the present invention.

FIG. 7 is a front view of the needle-mounted outer cylinder of the present invention on which a cap is mounted.

FIG. 8 is a sectional view of the needle-mounted outer cylinder shown in FIG. 7 taken along a section line 8-8 in FIG. 7.

FIG. 9 is an explanatory view for explaining a needle fixing process to be performed in a method of producing the needle-mounted outer cylinder of the present invention.

FIG. 10 is an explanatory view for explaining the needle fixing process in the method of producing the needle-mounted outer cylinder of the present invention.

FIG. 11 is an explanatory view for explaining a needle fixing process to be performed in a method of producing a needle-mounted outer cylinder of another embodiment of the present invention.

FIG. 12 is an explanatory view for explaining a welding process of welding a needle tube to an outer cylinder of another embodiment in the method of producing the needle-mounted outer cylinder of the present invention.

FIG. 13 is a graph showing an example of a mode of imparting energy to a needle tube in the method of producing the needle-mounted outer cylinder of the present invention.

FIG. 14 is a graph showing a heat-generated state of the needle tube when energy is imparted thereto.

### DETAILED DESCRIPTION

The needle-mounted outer cylinder disclosed here is described below with reference to several embodiments shown in the drawings.

A needle-mounted outer cylinder 1 disclosed here is composed of a metal needle tube 3 having a lumen penetrating therethrough from its distal end to its proximal end and an outer cylinder 2, made of thermoplastic resin, which has a distal part 22 having a needle insertion hole 41 into which a proximal portion of the needle tube 3 is inserted. The proximal portion 31 of the needle tube 3 is bonded to the outer cylinder with a melt-bond portion 45 formed inside the needle insertion hole 41 by melting an inner surface of the needle insertion hole 41 of the outer cylinder 2. Before the proximal portion 31 of the needle tube 3 is bonded to the outer cylinder 2, the needle insertion hole 41 has an inner diameter a little larger than an outer diameter of the proximal portion 31 of the needle tube 3. The melt-bond portion 45 is positioned at a side distal by a predetermined length from a proximal end of the needle insertion hole 41 and is formed by partly melting the inner surface of the needle insertion hole 41 and by filling a gap between an outer surface of the proximal portion 31 of the needle tube 3 and the inner surface of the needle insertion hole with a portion of the inner surface of the needle insertion hole which has melted and flowed down toward a proximal side of the needle insertion hole. The distal part 22 of the outer cylinder 2 does not substantially have bubbles to be formed when the melt-bond portion 45 is formed.

The needle-mounted outer cylinder disclosed here is used to inject a medicine into a living body by puncturing a needle tip into the surface of skin.

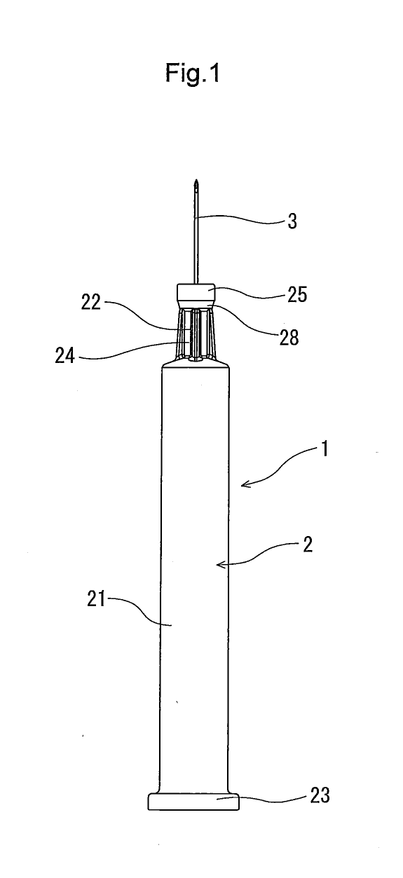

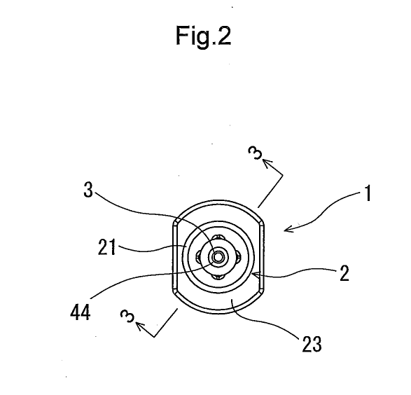

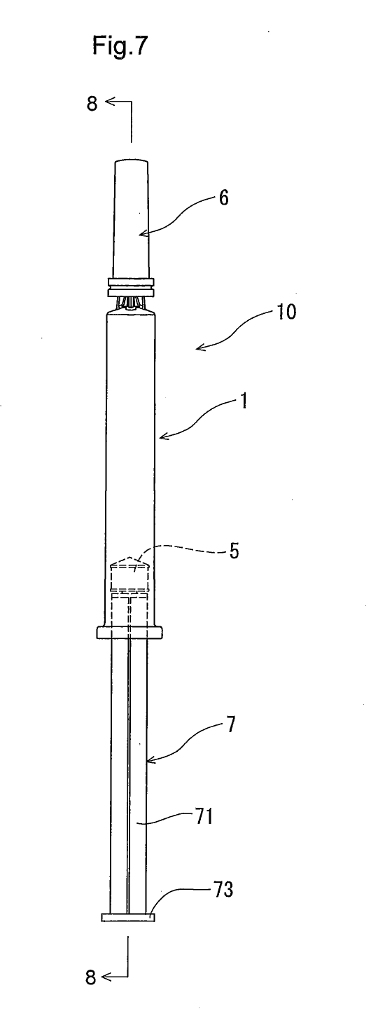

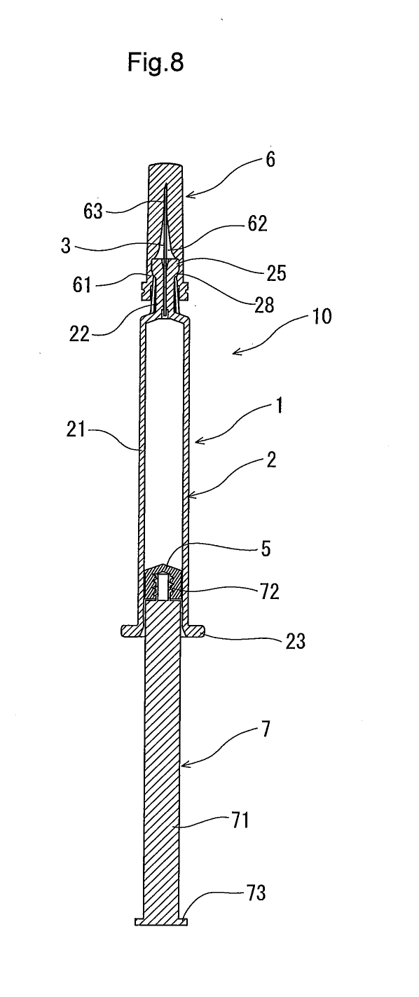

As shown in FIGS. 1 through 4, the needle-mounted outer cylinder 1 of this embodiment has the outer cylinder 2 and the needle tube 3 fixed to the inside of the distal part of the outer cylinder 2. As shown in FIGS. 7 and 8, a cap 6 is mounted on the needle-mounted outer cylinder 1.

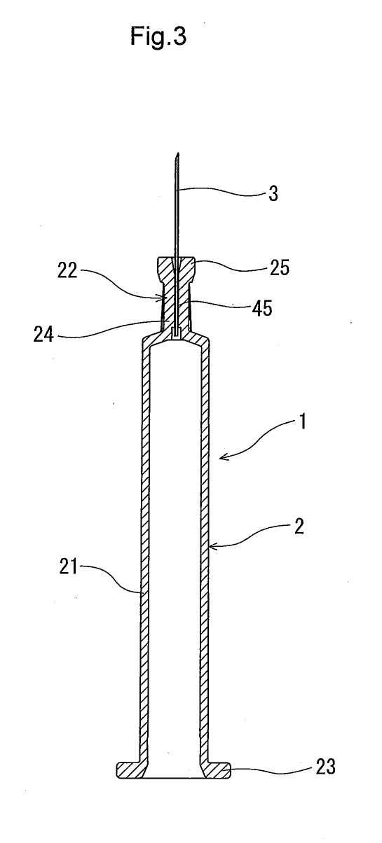

As shown in FIGS. 1 and 3, needle tubes of 27G to 30G (outer diameter: 0.41 to 0.31 mm) conforming to the ISO standard for a medical needle tube (ISO 9626: 1991/Amd. 1:2001(E)) are used as the needle tube 3.

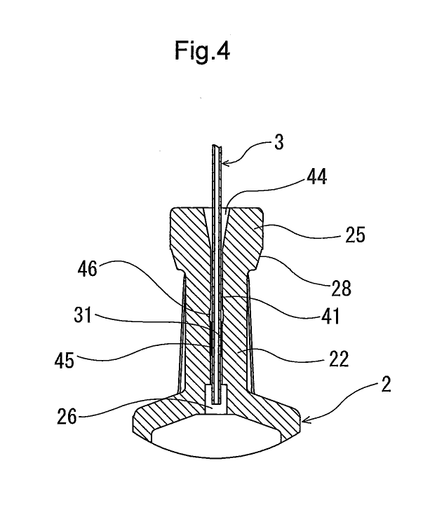

The needle tube 3 has the lumen penetrating therethrough from its distal end to its proximal end. The needle tube 3 has a needle tip 32 to be punctured into the living body at its distal end. The needle tip 32 having a blade surface is formed at an acute angle. A distal end portion of the needle tube 3 including the needle tip 32 projects from the distal end of the distal part 22 of the outer cylinder 3. A proximal end portion of the needle tube 3 is accommodated inside the needle insertion hole 41 of the outer cylinder 2. In this embodiment, the proximal end of the needle tube 3 penetrates through the needle insertion hole and reaches the inside of the outer cylinder 2 as seen in FIG. 4.

The proximal side portion of the needle tube 3 is positioned inside the needle insertion hole 41. It is preferable that an outer surface of a portion of the needle tube 3 to be fixed to the outer cylinder is a rough surface formed by blast treatment. By so doing, when the needle tube 3 is bonded to the outer cylinder 2 by welding, softened resin enters into concaves and convexes of the rough surface of the needle tube 3. Thereby it is possible to join the needle tube 3 with the outer cylinder 2 at a high joint strength. Further, because the softened resin enters into the concaves and the convexes of the rough surface of the needle tube 3, the needle-mounted outer cylinder of the present invention has an improved liquid-tightness. Thus the needle-mounted outer cylinder can be preferably used for a syringe.

As materials for the metal needle tube 3, stainless steel is preferable. But the materials for fabricating the metal needle tube 3 are not limited to stainless steel. It is possible to use aluminum, aluminum alloys, titanium, titanium alloys, and other metals. As the needle tube 3, it is possible to use not only a straight needle conforming to the ISO standard, but also a needle having larger or a smaller outer and inner diameters than those mentioned above. More specifically, in addition to needles of 31G or 32G, it is possible to use a tapered needle in which at least one portion is tapered. The needle tube 3 may have a polygonal configuration in addition to a circular configuration in its cross-sectional configuration. A coating agent consisting of silicone resin or fluororesin may be applied to the surface of the needle tip 32 of the needle tube 3.

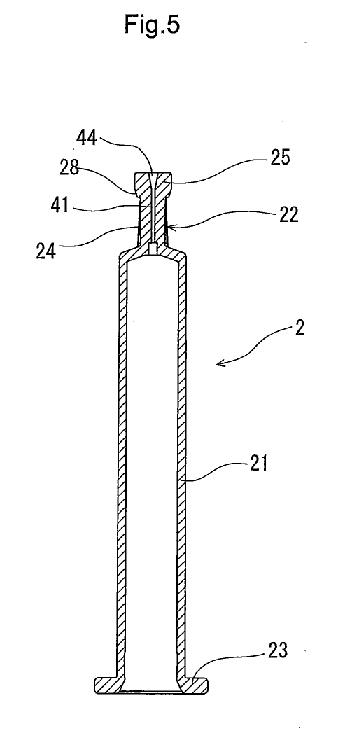

The outer cylinder 2 is described below. As shown in FIG. 5, the outer cylinder 2 has a main body part 21 in which a medicine is filled and a distal part 22 having the needle insertion hole 41. The main body part 21 has an internal accommodation portion and is formed substantially cylindrically. The main body part 21 has a flange 23 formed at its rear-end side (proximal end) in its axial direction.

The distal part 22 has a distal bulged portion 25, in which the outer dimension or outer diameter of the distal part 22 is enlarged relative to the immediately adjacent portion of the distal part 22, and a tubular portion 24 connecting the distal bulged portion 25 and the distal end of the main body part 21 to each other. The distal part 22 has the needle insertion hole 41 extending into the main body part 21 from the distal end thereof. It is favorable to form the inner diameter of the needle insertion hole 41 larger than the outer diameter of the needle tube 3 by 0.02 mm to 0.14 mm and more favorable by 0.02 mm to 0.09 mm. In the case where the needle tube 3 of 27G to 30G is used, by setting the inner diameter of the needle insertion hole 41 at 0.43 mm to 0.45 mm, the gap between the needle insertion hole 41 and the needle tube 3 can be set as described above. When any needle tube 3 whose outer diameter at the position of a melt-bond portion corresponds to 27 to 30G is used, it is possible to secure the joint strength of the needle tube 3 and prevent the needle tube 3 joined with the outer cylinder from tilting more than a predetermined angle after the needle tube is joined with the outer cylinder by setting the inner diameter of the needle insertion hole 41 to the above-described range. It is preferable that the thickness between the outer surface of the distal part 22 of the outer cylinder 2 and the needle insertion hole 41 is 1.2 mm to 1.5 mm. By setting the above-described thickness to the above-described range, energy can be efficiently imparted to the needle tube, and the outer surface of the distal part thereof does not easily melt. Thereby after the needle tube is joined with the outer cylinder, the outer cylinder has a high strength. Further it is possible to secure the joint strength of the needle tube 3 and prevent the needle tube 3 joined with the outer cylinder from tilting more than the predetermined angle.

As shown in FIG. 4, a distal end portion of the needle insertion hole 41 is formed as a distal diameter enlarged portion 44 having an inner diameter larger than the inner diameter of other portions of the needle insertion hole 41. This construction of the needle insertion hole allows the proximal portion 31 of the needle tube to be easily inserted into the needle insertion hole 41. The proximal end of the needle insertion hole 41 is formed as a proximal diameter enlarged portion 26 possessing an enlarged inner diameter relative to the immediately adjacent portion of the needle insertion hole 41. The proximal end of the needle tube 3 enters into the proximal diameter enlarged portion 26 and does not reach the inside of the main body part 21.

In this embodiment, an example in which the main body part 21 is formed approximately cylindrically is described. But the main body part 21 may be hollow square columnar or hollow hexagonal columnar.

As materials to form the outer cylinder 2, various resins such as polyvinyl chloride, polyethylene, polypropylene, cyclic polyolefin, polystyrene, poly-(methylpentene-1), polycarbonate, acrylic resin, acrylonitrile-butadiene-styrene copolymer, polyester such as polyethylene terephthalate, butadiene-styrene copolymer, and polyamide (for example, nylon 6, nylon 66, nylon 610, and nylon 12) can be used. Of these resins, it is preferable to use the polypropylene, the cyclic polyolefin, and the poly-(methylpentene-1). It is preferable that materials to form a joining member 4 and the outer cylinder 2 are substantially transparent to secure visibility of the inside thereof.

As the cyclic polyolefin, it is possible to preferably use cyclic polyolefin formed from components obtained by ring-opening metathesis polymerization of norbornene or norbornene derivatives, cyclic olefin polymer (COP) which is a hydrogenated product of the cyclic polyolefin, and cyclic olefin copolymer (COC) formed by copolymerization of norbornene and ethylene. In the cyclic polyolefin, in consideration of injection moldability in molding a material into an outer cylinder and the formability of the melt-bond portion in joining the needle tube with the outer cylinder, it is preferable to use cyclic polyolefins having the following MFR (melt flow rate) and glass transition temperature. That is, the MFR is favorably in the range of 10 to 40 g/10 minutes and more favorably in the range of 10 to 35 g/10 minutes. When the MFR is not less than 10 g/10 minutes, the flowability of the cyclic polyolefin does not become too low and the resin is capable of sufficiently entering into the concave-convex surface. Thus the cyclic polyolefin allows the needle tube to be joined with the outer cylinder at a high joining strength. In the case where resin having the MFR lower than 10 g/minute is used, it is possible to obtain a desired joining strength by pressurizing the outer cylinder to press the needle tube within a range not departing from the gist of the present invention, although it depends on the degree of the MFR having a low MFR. In the case where the MFR is not more than 40 g/minute, the flowability of the resin does not become too high, and melted resin stays at a position where the melt-bond portion is to be formed. Therefore it is possible to form a satisfactory melt-bond portion. The glass transition temperature is favorably in the range of 100 to 160 degrees C. Considering a case where the needle-mounted outer cylinder of the present invention is sterilized by using a high pressure steam in an overkill condition, the glass transition temperature is more favorably in the range of 125 to 160 degrees C.

As the COP to be used here, ZEONEX (registered trademark, produced by Nippon Zeon Co., Ltd.) and Daikyo Resin CZ (produced by Daikyo Seiko, Ltd.) are examples. As the COC, APEL (registered trademark, produced by Mitsui Chemicals Co., Ltd.), TOPAS (registered trademark, produced by Topas Advanced Polymers Gesellschaft mit beschrankter Haftung) are examples.

As shown in FIGS. 1 through 4, the needle tube 3 is welded to the outer cylinder 2 by using a production method described later to construct the needle-mounted outer cylinder 1. As shown in FIG. 4, the needle tube 3 is bonded to the outer cylinder 2 with the melt-bond portion 45 formed at a position disposed at a side proximal by a predetermined length or distance from the distal end of the needle insertion hole 41 of the distal part 22 of the outer cylinder 2 and at a position disposed at a side distal from the proximal end of the needle insertion hole 41 by a predetermined length or distance. More specifically, the proximal portion 31 of the needle tube 3 is bonded to the outer cylinder 2 at the position disposed at the side distal from the proximal end of the needle insertion hole 41 by the predetermined length or distance. The proximal end of the melt-bond portion 45 is formed or located at a position disposed at a side distal by a relatively short distance from the distal end of the proximal diameter enlarged portion 26 of the needle insertion hole 41. The distal end of the melt-bond portion 45 is formed or located at a position disposed at a side proximal from the distal inner diameter enlarged portion by a predetermined length or distance. In this embodiment, the needle insertion hole has an inner diameter enlarged portion (inner diameter enlarged portion formed of melted surface) 46 formed of a melted surface extended to a side distal from the distal end of the melt-bond portion 45 by a relatively short distance. The distal part 22 of the outer cylinder 2 including the melt-bond portion 45 does not substantially have bubbles to be generated owing to the formation of the melt-bond portion 45. To prevent the bubbles from being formed, it is preferable to form the melt-bond portion 45 by heating the needle tube to not less than the melting temperature of thermoplastic resin and less than the thermal decomposition temperature of the thermoplastic resin and thereafter solidifying the thermoplastic resin (without interposing other heating processes). The length of the melt-bond portion 45 is favorably 2.1 mm to 5.1 mm.

A syringe 10 on which the needle-mounted outer cylinder 1 disclosed here is mounted is described below.

As shown in FIG. 7, the syringe 10 has the needle-mounted outer cylinder 2, the cap 6 mounted on the distal part (needle part) of the outer cylinder 2, a gasket 5 accommodated inside the outer cylinder 2 and being slidable inside the outer cylinder, and a plunger 7 mounted on the gasket 5. The plunger 7 has a main body part 71, a gasket mounting part 72 formed at a distal end of the main body part 71, and a pressing part 73 formed at a proximal portion of the plunger. The gasket has a plunger mounting part which receives the gasket mounting part 72 of the plunger 6 and engages the gasket mounting part 72.

The cap 6 possesses a cylindrical configuration, open at a proximal portion 61 thereof in its axial direction, and closed at a distal end thereof in its axial direction. The cap 6 is formed of an elastic member such as rubber and elastomer. The cap 6 is mounted on the distal part 22 of the outer cylinder 2 in such a way as to cover the needle tip 32 of the needle tube 3 and the distal part 22 of the outer cylinder 2. As shown in FIG. 7, the needle tube 3 and the distal part 22 are inserted into a lumen portion 62 of the cap 6.

The inner diameter of the lumen portion 62 of the cap 6 is almost equally to the outer diameter of the distal bulged portion 25 of the distal part 22 or a little smaller than the outer diameter of the distal bulged portion 25. Therefore when the cap 6 is mounted on the distal part 22, the outer peripheral surface of the distal bulged portion 25 closely contacts the inner peripheral surface of the cap 6. Thereby a space covering the needle tube 3 projected from the outer cylinder 2 is closed with the distal bulged portion 25 and the inner peripheral surface of the cap 6. This construction prevents bacteria from sticking to the needle tip 32. At the same time, the needle tip holding portion 63 holds the needle tip 32.

An annular rib 61 formed on the inner peripheral surface of the cap 6 tightly contacts a constricted portion disposed at the boundary between the distal bulged portion 25 of the distal part 22 and a tapered fitting portion 28 of the distal part 22 by an elastic force of the annular rib 61. Owing to the engagement between the inner peripheral surface of the cap 6 and the constricted portion of the distal part 22, it is possible to prevent the cap 6 from being removed from the distal part 22 during delivery.

The method of producing the needle-mounted outer cylinder 1 is described below.

The production method disclosed here is to produce the needle-mounted outer cylinder 1 composed of the metal needle tube 3 having the lumen penetrating therethrough from its distal end to its proximal end and the outer cylinder 2, made of thermoplastic resin, which has the distal part 22 having the needle insertion hole 41 into which the proximal portion of the needle tube 3 is inserted. The proximal portion 31 of the needle tube 3 is bonded to the outer cylinder with the melt-bond portion 45 formed inside the needle insertion hole 41 by melting the inner surface of the needle insertion hole 41 of the outer cylinder 2.

The method of producing the needle-mounted outer cylinder disclosed here consists of execution of a melt-bond process of melting and bonding the proximal portion 31 of the needle tube 3 to the outer cylinder in a state in which the needle tube 3 is inserted into the needle insertion hole 41 of the outer cylinder 2 and in a state in which the outer cylinder 2 is erect. The melt-bond process is performed by imparting energy to the needle tube 3 at a portion of the needle tube 3 positioned at a side distal from the proximal end of the needle insertion hole 41 and from the proximal end of the needle tube to allow the needle tube 3 to generate heat so that resin which is positioned at a heat generation portion (energy imparting region) and forms the inner surface of the needle insertion hole 41 is melted, and thereafter by flowing the melted resin downward inside the needle insertion hole 41 to thereby fill the gap between the outer surface of the proximal portion 31 of the needle tube 3 and needle insertion hole 41.

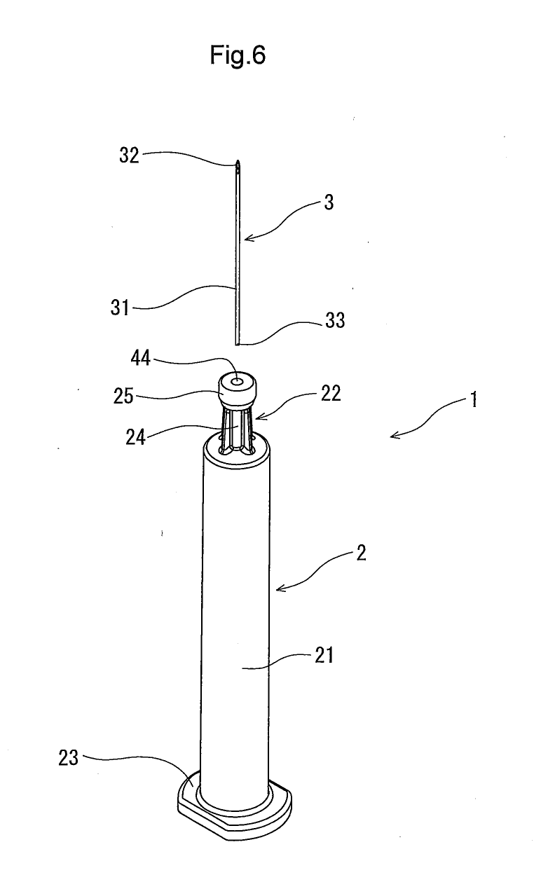

To produce the needle-mounted outer cylinder disclosed here, as shown in FIG. 6, the metal needle tube 3 and the outer cylinder 2 made of thermoplastic resin are prepared. The needle tube 3 is formed as a desired tubular body by press working a flat metal or swage processing a hollow pipe. The outer cylinder 2 is formed by injection-molding the thermoplastic resin.

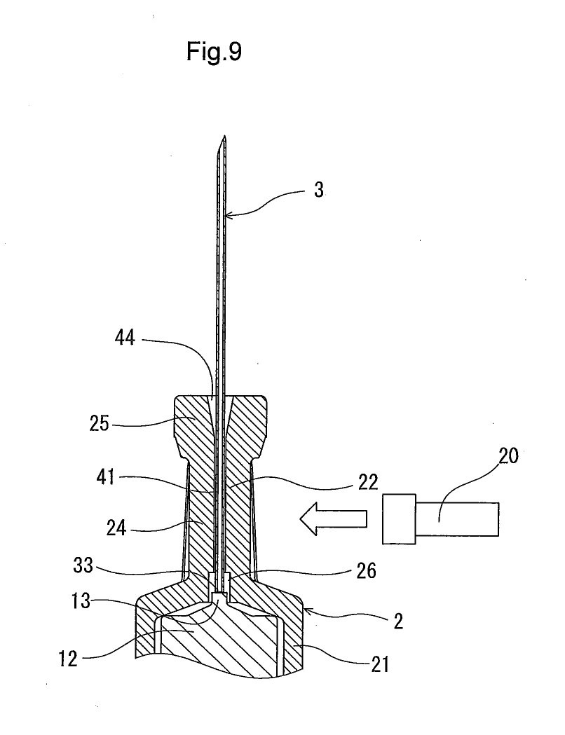

Thereafter the process of joining the needle tube 3 with the outer cylinder 2 is performed. In this joining process, initially the needle tube 3 is inserted into the distal inner diameter enlarged portion 44 of the needle insertion hole 41 of the outer cylinder 2 from the proximal side of the needle tube. That is, the proximal end of the needle tube 3 is inserted into the distal end of the needle insertion hole 41. As shown in FIG. 9, in this embodiment, the needle tube 3 is placed in position with respect to the outer cylinder 2 by using a needle supporting member 12. The needle supporting member 12 is disposed inside the internal accommodation portion of the main body part 21 and has a distally projecting supporting projection 13 which supports the proximal end 33 of the needle tube 3 at the upper end of the supporting projection 13. The needle supporting member 12 is so disposed that the supporting projection 13 of the needle supporting member 12 is positioned inside the proximal diameter enlarged portion 26 of the distal part 22. The proximal end of the needle tube 3 supported by or on the supporting projection 13 is positioned inside the proximal diameter enlarged portion 26 of the needle insertion hole 41 and does not project into the internal accommodation portion of the main body part 21. Therefore it is possible to decrease the dead volume inside the outer cylinder 2, decrease the amount of medicine remaining inside the outer cylinder 2, and prevent the distal end of the gasket from contacting the proximal end of the needle tube 3.

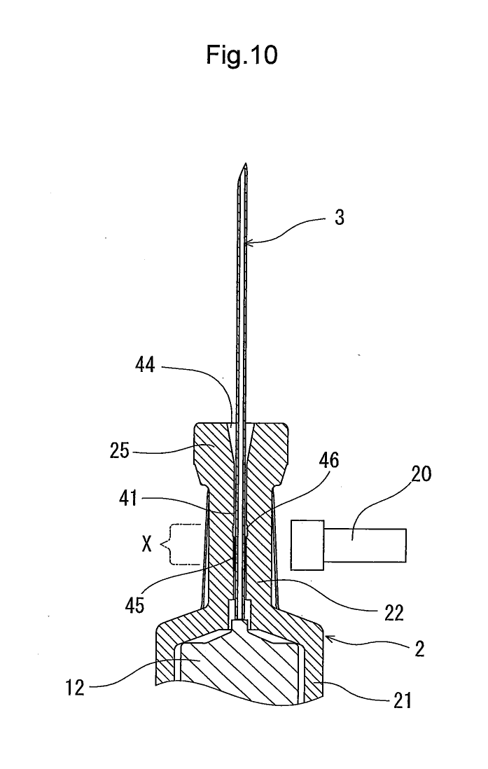

Thereafter the melt-bond process is performed. As shown in FIG. 10, in this embodiment, the melt-bond process is performed by using a semiconductor laser irradiation apparatus 20. The semiconductor laser irradiation apparatus 20 emits laser to a portion of the needle tube 3 corresponding to an energy imparting region X shown in FIG. 10. Thereby the portion of the needle tube 3 irradiated with the laser generates heat. Owing to the heat generated by the needle tube, the inner surface of the needle insertion hole 41 proximate to the outer surface of the heat generation portion of the needle tube 3 is heated by radiation (heat transfer) and melts.

Because the outer cylinder 2 is erect (i.e., is positioned vertically upright), the melted resin drops or falls by gravity. When the melted resin passes the energy imparting region (moves away from the heat generation portion of the needle tube), the melted resin hardens and fills the gap between the inner surface of the needle insertion hole and the outer surface of the needle tube to form the lower end portion of a welding portion. In this manner, the energy imparting operation finishes. The melt-bond process finishes when the resin hardens.

In the melt-bond process of the production method disclosed here, it is unnecessary to press a heating portion unlike the melt-bond process performed in the production method of the patent document 2. Because the heating portion is pressed, there is a possibility that deformation and distortion-caused crack may occur. On the other hand, in the method disclosed here, because it is unnecessary to perform a pressing operation, neither pressing-caused deformation nor pressing-caused crack occurs. Thus it is preferable to carry out the melt-bond process in such a way that the outer surface of the distal part 22 of the outer cylinder 2 does not reach the melting temperature of the resin and in a state in which a stress loading is not applied to the distal part 22 from the outer surface thereof.

In the needle-mounted outer cylinder 1 formed by carrying out the production method disclosed here, as shown in FIG. 4, the inner diameter enlarged portion 46 is formed at a position inside the distal part of the outer cylinder corresponding to the distal portion of the energy imparting region X owing to the melting and flow-down of the resin.

It is preferable that the melt-bond process has a needle tube heating process of allowing the needle tube to be heated and to generate heat in such a way that the thermoplastic resin heating temperature at the heat generation portion (energy imparting region) X becomes not less than the melting temperature of the thermoplastic resin and less than the thermal decomposition temperature of the thermoplastic resin. By performing this process, it is possible to prevent a bubble-holding portion from being formed inside the thickness of the distal part 22 of the outer cylinder 2 and outside the melt-bond portion 45.

It is possible to perform the melt-bond process by heating the entire needle tube in such a way that the thermoplastic resin heating temperature becomes not less than the melting temperature of the thermoplastic resin and less than the thermal decomposition temperature of the thermoplastic resin. To securely fix the needle tube to the outer cylinder in this method, a certain period of time is necessary to impart energy to the needle tube. Thus the working period of time is long. In addition, there is a possibility that the outer surface side of the energy imparting region may be heated and softened beyond the inner surface of the needle insertion hole of the outer cylinder.

It is preferable for the melt-bond process to carry out an initial heating process of allowing the needle tube to generate heat in such a way that the thermoplastic resin heating temperature at the heat generation portion (energy imparting region) X exceeds the thermal decomposition temperature of the thermoplastic resin and a final heating process of allowing the needle tube to generate heat in such a way that the thermoplastic resin heating temperature at the heat generation portion becomes not less than the melting temperature of the thermoplastic resin and less than the thermal decomposition temperature of the thermoplastic resin, after the execution of the initial heating process finishes.

By doing so, it is possible to fix the needle tube to the outer cylinder securely and in a short period of time and prevent the bubble-holding portion from being formed inside the thickness of the distal part 22 of the outer cylinder 2 and outside the melt-bond portion 45.

In the initial heating process (strong heating process), it is preferable to allow the needle tube to generate heat in such a way that the temperature of the resin at the heating portion becomes not less than the thermal decomposition temperature of the resin and not more than +25% of the thermal decomposition temperature of the resin. In the initial heating process (strong heating process), it is particularly preferable to allow the needle tube to generate heat in such a way that the temperature of the resin at the heating portion becomes not less than the thermal decomposition temperature of the resin by not less than +5% and not more than +20% of the thermal decomposition temperature of the resin. It is also preferable to perform the initial heating process (strong heating process) in such a way that the outer surface of the distal part of the outer cylinder does not reach the melting temperature of the resin. More specifically, by arranging the intensity of energy to be imparted to the needle tube and an energy imparting period of time, it is possible to accomplish the execution of the initial heating process. It is particularly preferable to perform the initial heating process by imparting a high energy to the needle tube in a short period of time. A portion of the needle insertion hole confronting (proximate to) the heating portion of the needle tube is strongly heated. Thus the resin at this portion securely melts and flows downward.

More specifically, in the initial heating process (strong heating process), it is preferable to set the output of the semiconductor laser irradiation apparatus 20, the laser focus diameter, the length of the energy imparting region (heating region), and the laser irradiation period of time to 10 W to 20 W, φ2 to 5 mm, +0.1 mm (2.1 mm to 5.1 mm) of the laser focus diameter, and 0.5 seconds to 1.5 seconds respectively.

In the final heating process, it is preferable to heat the needle tube in such a way that the temperature of the resin at the heating portion becomes not more than the thermal decomposition temperature of the thermoplastic resin and not less than the melting temperature of the thermoplastic resin. It is preferable to carry out the final heating process in such a way that the temperature of the outer surface of the distal part of the outer tube does not reach the melting temperature of the resin. More specifically, by arranging the intensity of the energy to be imparted to the needle tube and the energy imparting period of time, it is possible to perform the execution of the final heating process. For example, it is preferable to carry out the final heating process by imparting energy lower than that used in the initial heating process to the needle tube for a predetermined period of time. In the final heating process, it is preferable to allow the needle tube to generate heat in such a way that the temperature of the resin at the heating portion becomes lower than the thermal decomposition temperature of the resin by 5% to 20%. In the final heating process, it is preferable to allow the needle tube to generate heat in such a way that the temperature of the resin at the heating portion becomes higher than the melting temperature of the resin by at least 60 degrees C.

In the final heating process, it is preferable to maintain a laser focus diameter and the length of an energy imparting region (heating region) in the initial heating process and set the output of the semiconductor laser irradiation apparatus 20 to 40% to 60% of the output in the initial heating process. More specifically, it is preferable to set the output of the semiconductor laser irradiation apparatus to 5 W to 20 W. It is also preferable to set an irradiation period of time of the laser to 0.5 to 2.5 seconds.

It is preferable to set the total of the period of time required to perform the initial heating process and that required to perform the final heating process to one to five seconds. An intermediate heating process may be performed at a temperature not more than the thermal decomposition temperature of the resin between the execution of the initial heating process and that of the final heating process. The intermediate heating process may be performed at a temperature not more than the melting temperature of the resin.

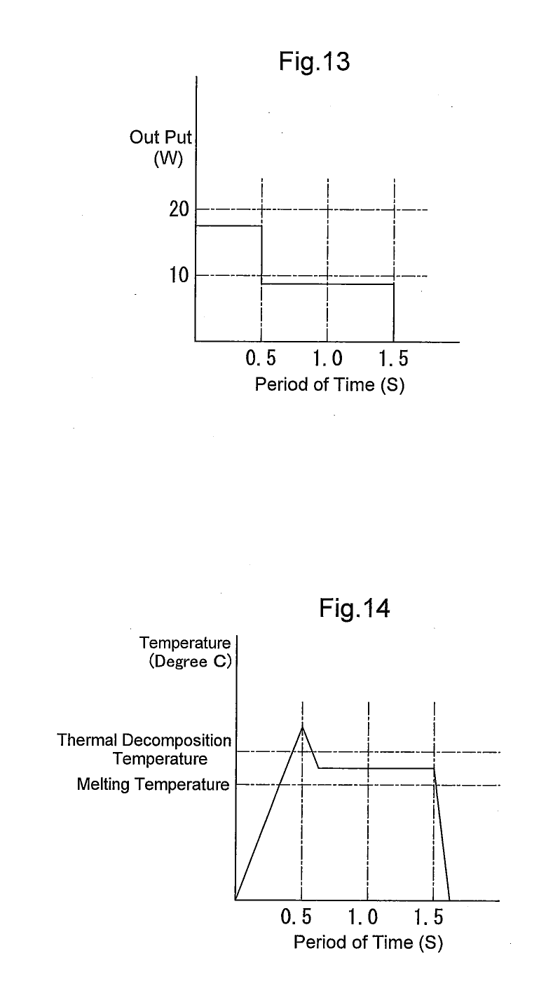

As the output imparting mode in which the semiconductor laser irradiation apparatus heats the needle tube, it is preferable to apply laser to the needle tube as shown in FIG. 13. In this example, initial heating is performed by applying the laser to the needle tube at an output of 20 W for 0.5 seconds. Thereafter the laser is applied to the needle tube for one second by decreasing the output to 10 W. As shown in FIG. 14, in imparting the energy to the needle tube as in the case of this example, after the needle tube is initially heated to quickly increase the temperature thereof to not less than the thermal decomposition temperature of the resin, the temperature of the needle tube is maintained at a temperature less than the thermal decomposition temperature of the resin and not less than the melting temperature thereof for a predetermined period of time. Thereafter the temperature of the needle tube returns to a normal temperature.

In the above description, the initial heating process in a melting/heating process is performed at a first output level (high level), while the final heating process is performed at a second output level (low level) lower than the first output level. But alternatively, the final heating process may be performed by gradually lowering the first output level set in the initial heating process to allow the needle tube to generate heat.

By adjusting the position at which the melt-bond portion 45 is to be formed, the flexibility (deflection) of the needle tube can be controlled. For example to prevent the needle tube from kinking (bending) by allowing the needle tube to easily bend, it is preferable to form the melt-bond portion 45 at a position proximate to the proximal inner diameter enlarged portion 26 of the needle insertion hole 41 in such a way that the position at which the melt-bond portion is to be formed does not reach the proximal diameter enlarged portion 26. In the case where the bending of the needle tube is not desired, it is preferable to form the melt-bond portion at a position where the distal end of the melt-bond portion 45 is proximate to the distal diameter enlarged portion 44 of the needle insertion hole 41.

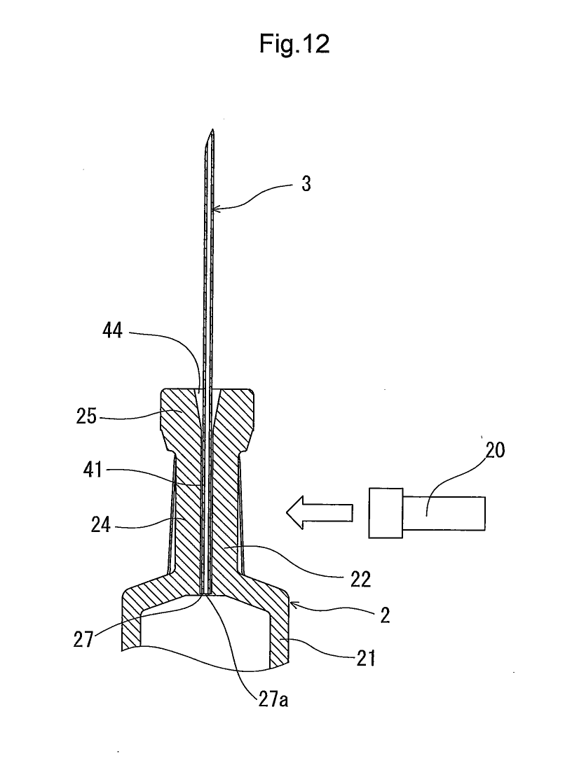

In the above-described embodiment, in the process of inserting the needle tube 3 into the needle insertion hole, the needle tube 3 is placed in position by using the needle supporting member 12 separate from the outer cylinder 2. But the needle tube 3 may be placed in position by providing the outer cylinder with a construction different from the above-described one. Like another embodiment shown in FIG. 12, the needle tube 3 may be placed in position by bringing the needle tube into contact with a needle stop portion 27 formed inside the distal part 22 of the outer cylinder 2. The needle stop portion 27 is formed of an annular rib projected inward (radially inward) from the inner surface of the proximal end of the needle insertion hole 41. A communication hole 27a positioned at a central portion of the needle stop portion 27 allows communication between the needle insertion hole 41 and the main body part 21. Therefore the lumen of the needle tube 3 placed in position with the needle tube in contact with the needle stop portion 27 communicates with the internal accommodation portion of the main body part 21 through the communication hole 27a. In this manner, the needle tube 3 is placed in position with respect to the outer cylinder 2.

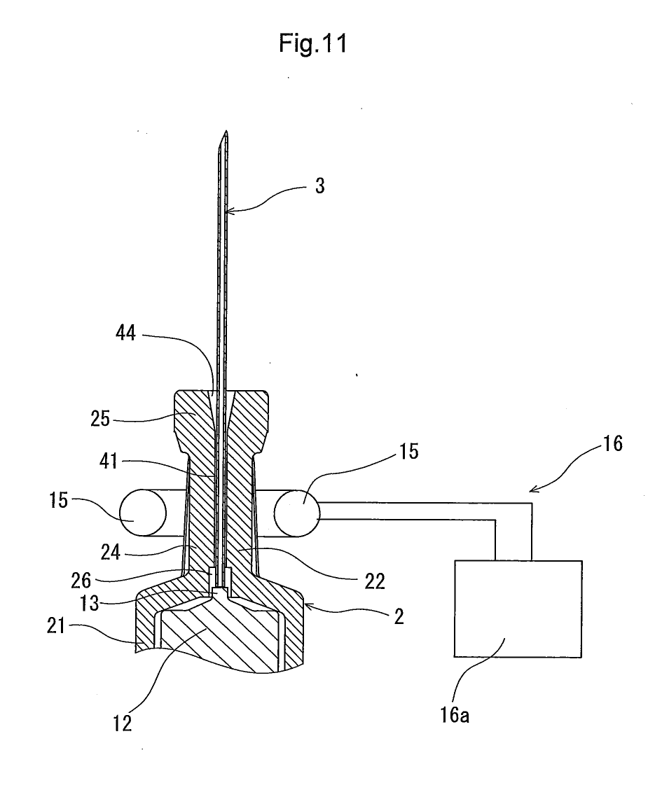

As an energy imparting means to be used in the melt-bond process of the production method disclosed here, it is preferable to use the above-described semiconductor laser irradiation apparatus. But as the energy imparting means, like an embodiment shown in FIG. 11, a high frequency induction heating apparatus 16 may be used. The high frequency induction heating apparatus 16 has a work (working) coil 15 and a power supply 16a which applies an alternating current to the work coil 15. Upon application of the alternating current from the power supply 16a to the work coil 15, a magnetic field is generated on the periphery of the work coil 15 to generate an eddy current in the needle tube 3. Thereby the needle tube 3 is heated and generates heat.

### EXAMPLE

Outer cylinders (capacity: 1 mL) having a configuration as shown in FIGS. 5 and 6 were produced by using cyclic olefin polymer (COP produced by Nippon Zeon Co., Ltd.). The inner diameter of the needle insertion hole disposed at the distal part of the outer cylinder was 0.43 mm. Needle tubes made of stainless steel of 27G (outer diameter: about 0.41 mm) and 29G (outer diameter: about 0.34 mm) were used. As shown in FIGS. 9 and 10, to heat the needle tubes, a portion of each needle tube positioned at a side a little distal from the proximal portion thereof was irradiated by using the semiconductor laser irradiation apparatus in a state in which the outer cylinder was substantially vertically disposed and the needle supporting member 12 was disposed inside the outer cylinder. In the initial heating process (strong heating process), the output of the semiconductor laser irradiation apparatus, the laser focus diameter, and the laser irradiation period of time were set to 18 W, φ3 m, and 0.5 seconds respectively. After the execution of the initial heating process finished, the final heating process was performed in succession. The final heating process was performed in a manner similar to that of the initial heating process except that the output of the laser irradiation and the laser irradiation period of time were set to 7 W and two seconds respectively. After the execution of the final heating process finished, the outer cylinders were naturally cooled. In this manner, a plurality of the needle-mounted outer cylinders of an embodiment of the present invention was produced.

### EXPERIMENT

As a result of visual observation of the needle-mounted outer cylinder (27G was used. Example 1) and the needle-mounted outer cylinder (29G was used. Example 2) produced in the above-described example, the generation of bubbles at the distal part thereof and distortion and deformation of the distal part thereof were not recognized. As a result of the measurement of the length of the melt-bond portion in the axial direction of the outer cylinder, the length of each needle-mounted outer cylinder was about 3 mm.

The pull-out strength of the needle of the needle-mounted outer cylinder of each of the examples 1 and 2 (six pieces respectively) was measured in accordance with JIS 3209, 2011. The pull-out strength of the needle of the needle-mounted outer cylinder of the Example 1 was 70N to 110N. The pull-out strength of the needle of the needle-mounted outer cylinder of the Example 2 was 50N to 90N.

The needle-mounted outer cylinder disclosed here is as described below.
(1) A needle-mounted outer cylinder comprises a metal needle tube having a lumen penetrating therethrough from a distal end thereof to a proximal end thereof and an outer cylinder, made of thermoplastic resin, which has a distal part having a needle insertion hole into which a proximal portion of said needle tube is inserted; and said proximal portion of said needle tube is bonded to said outer cylinder with a melt-bond portion formed inside said needle insertion hole by melting an inner surface of said needle insertion hole of said outer cylinder, wherein said needle insertion hole has an inner diameter a little larger than an outer diameter of said proximal portion of said needle tube; said melt-bond portion is positioned at a side distal by a predetermined length from a proximal end of said needle insertion hole and is formed by partly melting said inner surface of said needle insertion hole, by flowing down toward a proximal side of said needle insertion hole and by filling a gap between an outer surface of said proximal portion of said needle tube and said inner surface of said needle insertion hole with a portion of said inner surface of said needle insertion hole; and said distal part of said outer cylinder does not substantially have bubbles to be formed when said melt-bond portion is formed.

In the needle-mounted outer cylinder, because the needle tube is securely fixed to the needle insertion hole formed at the distal part of the outer cylinder, the needle tube does not separate from the outer cylinder during use.
(2) A needle-mounted outer cylinder according to the above (1), wherein said melt-bond portion is formed by heating said thermoplastic resin in such a way that a temperature of said thermoplastic resin becomes not less than a melting temperature thereof and less than a thermal decomposition temperature thereof and thereafter solidifying said thermoplastic resin.
(3) A needle-mounted outer cylinder according to the above (1) or (2), wherein said needle insertion hole has a diameter enlarged portion formed of a melted surface extended from said distal end of said melt-bond portion.

The method of producing the needle-mounted outer cylinder disclosed here is as follows:
(4) A method of producing a needle-mounted outer cylinder comprises a metal needle tube having a lumen penetrating therethrough from a distal end thereof to a proximal end thereof and an outer cylinder, made of thermoplastic resin, which has a distal part having a needle insertion hole into which a proximal portion of said needle tube is inserted; and said proximal portion of said needle tube is bonded to said outer cylinder with a melt-bond portion formed inside said needle insertion hole by melting an inner surface of said needle insertion hole of said outer cylinder, said method of producing said needle-mounted outer cylinder consists of execution of a melt-bond process of melting and bonding said proximal portion of said needle tube to said outer cylinder in a state in which said needle tube is inserted into said needle insertion hole of said outer cylinder and in which said outer cylinder is erect; and said melt-bond process being performed by imparting energy to said needle tube at a portion thereof positioned at a side distal from a proximal end of said needle insertion hole and from said proximal end of said needle tube to allow said needle tube to generate heat so that resin positioned at a heat generation portion and forming said inner surface of said needle insertion hole is melted, and thereafter by flowing said melted resin downward inside said needle insertion hole to thereby fill a gap between an outer surface of said proximal portion of said needle tube and said needle insertion hole. According to the production method of the present invention, it is possible to easily and securely produce the needle-mounted outer cylinder which allows a needle tube insertion work to be performed easily by setting into the needle insertion hole formed at the distal part of the outer cylinder larger than the outer diameter of the needle tube and which hardly generates foaming at the melt-bond portion and foaming-caused defective fixing of the needle tube to the outer cylinder.
(5) A method of producing a needle-mounted outer cylinder according to the above (4), wherein said melt-bond process includes a needle tube heating process of allowing said needle tube to generate heat in such a way that a thermoplastic resin heating temperature at said heat generation portion becomes not less than a melting temperature of said thermoplastic resin and less than a thermal decomposition temperature thereof; and a solidifying process of solidifying said melted resin subsequently to finish of execution of said needle tube heating process.
(6) A method of producing a needle-mounted outer cylinder according to the above (4), wherein said melt-bond process consists of an initial heating process of allowing said needle tube to generate heat in such a way that a thermoplastic resin heating temperature at said heat generation portion exceeds a thermal decomposition temperature of said thermoplastic resin and execution of a final heating process of allowing said needle tube to generate heat in such a way that said thermoplastic resin heating temperature at said heat generation portion becomes not less than a melting temperature of said thermoplastic resin and less than said thermal decomposition temperature thereof, after execution of said initial heating process finishes.
(7) A method of producing a needle-mounted outer cylinder according to the above (6), wherein said needle tube is allowed to generate heat by irradiating said needle tube with laser or high-frequency induction heating; and said initial heating process is performed by irradiating said needle tube with laser or high-frequency induction heating at a first output level for a predetermined period of time; and said final heating process is performed by irradiating said needle tube with laser or high-frequency induction heating for a predetermined period of time at a level lower than said first output level.
(8) A method of producing a needle-mounted outer cylinder according to the above (7), wherein said final heating process is performed to allow said needle tube to generate heat at a second output level lower than said first output level.
(9) A method of producing a needle-mounted outer cylinder according to the above (7), wherein said final heating process is performed to allow said needle tube to generate heat by gradually lowering said first output level.
(10) A method of producing a needle-mounted outer cylinder according to any one of the above (4) through (9), wherein said melt-bond process is performed in such a way that an outer surface of said distal part of said outer cylinder does not reach a melting temperature of said resin and in a state in which a stress loading is not applied to said distal part of said outer cylinder from said outer surface thereof.

The detailed description above describes features and aspects of a needle-mounted outer cylinder and a method of producing the needle-mounted outer cylinder representing examples of the needle-mounted outer cylinder and method disclosed here. The present invention is not limited, however, to the precise embodiments and variations described. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

1. A needle-mounted outer cylinder comprising:
a metal needle tube and an outer cylinder, the needle tube possessing a proximal portion;
the outer cylinder being made of thermoplastic resin, the outer cylinder including a distal part having a needle insertion hole;
the proximal portion of said needle tube is bonded to said outer cylinder by way of a melt-bond portion inside said needle insertion hole formed by melting an inner surface of said needle insertion hole;
said needle insertion hole possessing an inner diameter larger than an outer diameter of said proximal portion of said needle tube;
said melt-bond portion being positioned along the needle insertion hole at a position distal of a proximal end of said needle insertion hole and being formed by partly melting the resin forming said inner surface of said needle insertion hole, by causing the partly melted resin to flow down toward a proximal end of said needle insertion hole and by filling a gap between an outer surface of said proximal portion of said needle tube and said inner surface of said needle insertion hole with a portion of said partly melted resin.

2. A needle-mounted outer cylinder according to claim 1, wherein said melt-bond portion is formed by heating said thermoplastic resin in such a way that a temperature of said thermoplastic resin becomes not less than a melting temperature of the thermoplastic resin and less than a thermal decomposition temperature of the thermoplastic resin, and thereafter solidifying said thermoplastic resin.

3. A needle-mounted outer cylinder according to claim 2, wherein said needle insertion hole possesses an inner diameter, the needle insertion hole including an inner diameter enlarged portion at which the inner diameter of the needle insertion hole is enlarged relative to an immediately adjoining part of the needle insertion hole, the inner diameter enlarged portion extending from a distal end of said melt-bond portion.

4. A needle-mounted outer cylinder according to claim 2, wherein said needle insertion hole possesses an inner diameter, the needle insertion hole including an inner diameter enlarged portion at which the inner diameter of the needle insertion hole is enlarged relative to an immediately adjoining part of the needle insertion hole, the inner diameter enlarged portion extending distally from a distal end of said melt-bond portion.

5. A needle-mounted outer cylinder according to claim 1, wherein said needle insertion hole possesses an inner diameter, the needle insertion hole including an inner diameter enlarged portion at which the inner diameter of the needle insertion hole is enlarged relative to an immediately adjoining part of the needle insertion hole, the inner diameter enlarged portion being positioned at a distal-most part of the outer cylinder.

6. A needle-mounted outer cylinder according to claim 1, wherein said needle insertion hole possesses an inner diameter, the needle insertion hole including an inner diameter enlarged portion at which the inner diameter of the needle insertion hole is enlarged relative to an immediately adjoining part of the needle insertion hole, the inner diameter enlarged portion being positioned at a distal-most part of the outer cylinder.

7. A needle-mounted outer cylinder according to claim 6, wherein the inner diameter enlarged portion of the needle insertion hole possesses a gradually tapering inner diameter.

8. A needle-mounted outer cylinder according to claim 1, wherein said needle insertion hole possesses a proximal inner diameter enlarged portion at which the inner diameter of the needle insertion hole is enlarged relative to an immediately adjoining part of the needle insertion hole, the proximal inner diameter enlarged portion being positioned proximal of a proximal-most end of the melt-bond portion.

9. A method of producing a needle-mounted outer cylinder comprising:
inserting a needle tube into a needle insertion hole of an outer cylinder, the needle tube possessing a proximal end, the needle insertion hole of the outer cylinder also possessing a proximal end and an inner surface facing the needle tube;
performing melt-bond processing while the needle tube is positioned in the needle insertion hole of the outer cylinder by imparting energy to a portion of said needle tube positioned distal from a proximal end of said needle insertion hole and distal from said proximal end of said needle tube to cause said needle tube to generate heat so that resin positioned at a heat generation portion and forming said inner surface of said needle insertion hole is melted to form melted resin; and thereafter
causing the melted resin to flow downward inside said needle insertion hole while the outer cylinder is positioned upright so that the melted resin fills a gap between an outer surface of a proximal portion of said needle tube and said needle insertion hole.

10. The method of producing a needle-mounted outer cylinder according to claim 9, wherein said melt-bond process includes a needle tube heating process in which said needle tube generates heat in such a way that the thermoplastic resin is heated to a heating temperature at said heat generation portion that is not less than a melting temperature of said thermoplastic resin and that is less than a thermal decomposition temperature of said thermoplastic resin; and a solidifying process of solidifying said melted resin subsequently to finish of execution of said needle tube heating process.

11. The method of producing a needle-mounted outer cylinder according to claim 9, wherein said melt-bond processing comprises an initial heating process in which said needle tube is heated to generate heat in such a way that the thermoplastic resin is heated to a heating temperature at said heat generation portion that exceeds a thermal decomposition temperature of said thermoplastic resin, and a final heating process in which said needle tube is heated to generate heat in such a way that said thermoplastic resin is heated to a heating temperature at said heat generation portion that is not less than a melting temperature of said thermoplastic resin and less than said thermal decomposition temperature of said thermoplastic resin, the final heating process being started after completing said initial heating process.

12. The method of producing a needle-mounted outer cylinder according to claim 11, wherein said needle tube is heated to generate the heat by irradiating said needle tube with a laser or with high-frequency induction heating; said initial heating process is performed by irradiating said needle tube with laser or high-frequency induction heating at a first output level for a predetermined period of time; and said final heating process is performed by irradiating said needle tube with laser or high-frequency induction heating for a predetermined period of time at a level lower than said first output level.

13. The method of producing a needle-mounted outer cylinder according to claim 12, wherein said final heating process is performed to allow said needle tube to generate heat at a second output level lower than said first output level.

14. The method of producing a needle-mounted outer cylinder according to claim 12, wherein said final heating process is performed to allow said needle tube to generate heat by gradually lowering said first output level.

15. The method of producing a needle-mounted outer cylinder according to claim 9, wherein said melt-bond process is performed in such a way that an outer surface of a distal part of said outer cylinder does not reach a melting temperature of said resin and in a state in which a stress loading is not applied to said distal part of said outer cylinder from an outer surface of said outer cylinder.

16. A method of producing a needle-mounted outer cylinder comprising:
inserting a proximal end of a needle tube into a distal end of a needle insertion hole disposed at a distal-most part of an outer cylinder so that a proximal portion of the needle tube is positioned inside the needle insertion hole while a distal portion of the needle tube projects distally beyond the distal-most part of an outer cylinder, the needle insertion hole of the outer cylinder also possessing a proximal end and an inner surface facing the needle tube;
applying energy to a portion of the needle tube located in the needle insertion hole of the outer cylinder, the portion of the needle tube to which the energy is applied being spaced distally from a proximal-most end of the needle insertion hole and distal from a proximal-most end of the, the energy being applied to the portion of the needle tube while the needle tube is vertically oriented, the energy being applied to the portion of the needle tube to heat the portion of the inner tube so that resin positioned at a heat generation portion and forming the inner surface of the needle insertion hole is melted to form melted resin; and
causing the melted resin to flow downward inside the needle insertion hole by gravity to fill a gap between an outer surface of a proximal portion of the needle tube and the needle insertion hole with the melted resin.

17. The method of producing a needle-mounted outer cylinder according to claim 16, wherein the imparting of the energy to the portion of the needle tube comprises irradiating the needle tube with a laser or with high-frequency induction heating.

18. The method of producing a needle-mounted outer cylinder according to claim 16, wherein the applying of the energy to the portion of the needle tube to heat the portion of the inner tube comprises: i) an initial heating process in which the energy is applied to the needle tube at a first output level; and ii) a final heating process in which the energy is applied to the needle tube at a second output level lower than the first output level.

19. The method of producing a needle-mounted outer cylinder according to claim 16, wherein the needle insertion hole possesses a proximal inner diameter enlarged portion at which the inner diameter of the needle insertion hole is enlarged relative to an immediately adjoining part of the needle insertion hole, and wherein the inserting of the proximal end of the needle tube into the distal end of the needle insertion hole includes positioning the needle tube so that the proximal end of the needle tube is located in the proximal inner diameter enlarged portion of the needle insertion hole.

20. The method of producing a needle-mounted outer cylinder according to claim 16, wherein the needle insertion hole possesses a proximal inner diameter enlarged portion at which the inner diameter of the needle insertion hole is enlarged relative to an immediately adjoining part of the needle insertion hole, and wherein the inserting of the proximal end of the needle tube into the distal end of the needle insertion hole includes positioning the needle tube so that a proximal-most end of the needle tube is located distal of the proximal inner diameter enlarged portion of the needle insertion hole.
